(19) United States
(12) Patent Application Publication
(10) Pub. No.: US 2004/0247631 A1
(43) Pub. Date: Dec. 9, 2004

(54) Increasing the skin-moisturizing properties of polyols

(75) Inventors: Rainer Kropke, Schenefeld (DE); Jens Nielsen, Henstedt-Ulzburg (DE); Anja Goppel, Hamburg (DE); Ariane Kranz, Hamburg (DE); Albrecht Dorschner, Hamburg (DE)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Beiersdorf AG

(21) Appl. No.: 10/790,910

(22) Filed: Mar. 1, 2004

Related U.S. Application Data
(63) Continuation of application No. PCT/EP02/09577, filed on Aug. 28, 2002.

(30) Foreign Application Priority Data
Sep. 1, 2001 (DE) 101 429 31.2

(51) Int. Cl. A61K 7/00

(52) U.S. Cl. 424/401

(57) ABSTRACT

The present invention is a cosmetic or dermatological formulation, comprising iminodisuccinic acid or a salt thereof and a polyol, and a method of applying same to the skin to moisturize the skin. The present invention also includes methods of increasing the skin-moisturizing properties of polyols by using iminodisuccinic acid or a salt thereof.

### CROSS-REFERENCE TO RELATED APPLICATIONS

This is a continuation application of PCT/EP02/09577, filed Aug. 28, 2002, which is incorporated herein by reference in its entirety, and also claims the benefit of German Priority Application No.101 42 931.2, filed Sep. 1, 2001.

### FIELD OF THE INVENTION

The present invention relates to cosmetic or dermatological formulations comprising iminodisuccinic acid or its salts as well as polyols, in addition to other active compounds, auxiliaries and additives, and to their use.

### BACKGROUND OF THE INVENTION

One of the most important tasks of cosmetic and dermatological formulations is moisturizing and moisture regulation of the skin. For this purpose, in addition to water as a constituent of all emulsions, so-called moisturizers are added to the formulations. Synthetic moisturizers are substitutes for the natural moisturizing factor (NMF), which comprises 40% of free amino acids, 12% of pyroglutamic acid, 12% of lactates, 7% of urea, 1.5% of uric acid and glucosamine, creatinine and various salts. In addition to hydrolyzed proteins, polyols (polyhydric alcohols) are used above all as synthetic moisturizers.

The most important representative of the polyols is glycerol (glycerin, 1,2,3-propanetriol), a colorless and odorless, sweet-tasting liquid. Glycerol has the following structure:

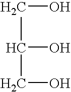

Another important representative of the polyols is sorbitol, a pentahydric alcohol which occurs in rowanberries and can be obtained synthetically by reduction of glucose.

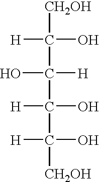

The moisture content of the skin can be determined by means of corneometric measurements. In these, the dielectric properties of the stratum corneum are investigated with the aid of a corneometer. The corneometer comprises a scatter capacitor, the capacity of which is (co)-determined by the dielectric properties of the stratum corneum. To determine how long the skin moisturization effected by a cosmetic or dermatological formulation lasts, the moisture content of the skin is determined under constant measuring conditions in each case before use and two hours after use of the cosmetic or dermatological formulation.

### SUMMARY OF THE INVENTION

A great disadvantage of the prior art is that the moisturizing action of cosmetic and dermatological formulations on the skin as a rule is only of short duration, so that the object of the present invention was to develop cosmetic or dermatological formulations which moisturize the skin over a longer period of time.

Surprisingly, the object is achieved by cosmetic or dermatological formulations comprising iminodisuccinic acid or its salts as well as polyols, in addition to other active compounds, auxiliaries and additives.

In this context, a 0.001 to 15 wt. %, advantageously 0.01 to 10 wt. %, very particularly preferably 0.05 to 5 wt. % strength iminodisuccinic acid or salts thereof is advantageous according to the invention, the weight data in each case relating to the total weight of the formulation.

The iminodisuccinic compound which is advantageous according to the invention in this context is the tetrasodium salt.

The concentration of polyols which is advantageous according to the invention is 3 to 65% by weight, and in particular 5 to 25% by weight, in each case based on the total weight of the formulation.

In this context, the polyols glycerol, sorbitol and butylene glycol are particularly advantageous according to the invention.

### DETAILED DESCRIPTION OF THE PREFERRED EMBODIMENTS

According to the invention, the iminodisuccinic acid or its salts are used to increase the skin-moisturizing action of polyols. According to the invention, the increase in the skin-moisturizing action of polyols is at least 25% if 2 hours have passed after application of the formulation to the skin and if the skin moisture content is determined as follows:

Corneometer: CM 825 apparatus from Courage & K., Cologne

Measurement conditions: 21° C.±1° C. and 50±5% atmospheric humidity at least 15 min acclimatization time

Measurement times: t₀=immediately before application of the formulation

t₁=2 hours after application of the formulation

The cosmetic or dermatological formulations according to the invention can advantageously be used as skin care products, as face care products and as sunscreen compositions.

In the context of the invention, “skin care products” are understood here as meaning, inter alia, skin creams, skin lotions, milks, ointments, oils, balsams and sera which are used for care of the skin.

Face care products are used as a special form of skin care products for care of facial skin. They are used in particular to prevent developing or to reduce already existing wrinkles and folds.

According to the invention, face care products also include decorative cosmetics, the main purpose of which is to change the color of skin and skin appendages (e.g. eyelashes, eyebrows).

Sunscreen compositions in the context of the invention are to be understood as meaning all forms of formulations which comprise at least one UV light protection filter. They furthermore include so-called “aftersun products.” These are intended to cool the skin after sunbathing and to improve its moisture retention capacity, the imparting of the cooling effect playing a central role. This cooling effect is as a rule achieved by large amounts of ethanol and water, which evaporates spontaneously when the formulation is spread on the skin. The preparations furthermore usually comprise moisturizing agents, such as glycerol or propylene glycol, and anti-inflammatory compounds, such as, for example, allantion, α-bisabolol, panthenol or aloe vera extract.

The following examples are intended to illustrate the present invention without limiting it. Unless stated otherwise, all the amounts data, contents and percentage contents are based on the weight and the total amount or on the total weight of the formulations.

### EXAMPLES

### W/O Emulsions

W/O emulsions

| | 1 | 2 | 3 | 4 | 5 |
|---|---|---|---|---|---|
| Triglycerol diisostearate | 1.0 | 0.5 | 0.25 | 2.0 | 3.0 |
| Diglycerol dipolyhydroxystearate | 1.0 | 1.5 | 1.75 | 3.0 | 2.0 |
| Paraffin oil | 12.5 | 10.0 | 8.0 | 5.0 | 17.5 |
| Vaseline | 8.0 | 6.0 | 5.0 | 12.0 | 2.5 |
| Hydrogenated coconut glycerides | 2.0 | 1.0 | 2.5 | 5.0 | 0.25 |
| Decyl oleate | 0.5 | 0.75 | 1.0 | 2.0 | 0.25 |
| Octyldodecanol | 0.5 | 1.0 | 0.75 | 3.0 | 0.25 |
| Aluminum stearate | 0.4 | 0.3 | 0.6 | 1.0 | 0.05 |
| Dicaprylyl carbonate | 0.1 | 0.05 | 0.15 | 0.5 | 1.0 |
| Hydrogenated castor oil | 0.5 | 0.75 | 1.0 | 2.5 | 5.0 |
| Iminodisuccinic acid | 0.5 | — | — | — | 0.1 |
| Magnesium sulfate | 0.5 | 0.6 | 0.5 | 0.7 | 1.0 |
| Glycerol | 3.0 | 5.0 | 10.0 | 15.0 | 1.5 |
| Tetrasodium iminosuccinate | — | 0.6 | 1.5 | 0.4 | — |
| Perfume | q,s. | q,s, | q,s, | q,s, | q,s, |
| Ethanol | 2.0 | — | 5.0 | — | — |
| Caprylic/capric acid triglyceride | 2.0 | 2.5 | 3.0 | 5.0 | 0.5 |
| Methyl paraben | 0.4 | 0.15 | 0.05 | 0.3 | 0.4 |
| Propyl paraben | 0.3 | 0.4 | 0.25 | 0.15 | — |
| Iodopropynyl butyl carbamates | — | — | 0.05 | — | 0.1 |
| Water | to 100 | to 100 | to 100 | to 100 | to 100 |
| | 6 | 7 | 8 | 9 | 10 |
| PEG-30 dipolyhydroxystearate | — | 0.5 | 0.25 | — | 3.0 |
| Lanolin alcohol | 1.0 | 1.5 | 1.75 | 3.0 | — |
| Paraffin oil | 12.5 | 10.0 | 8.0 | 5.0 | 17.5 |
| Vaseline | 8.0 | 6.0 | 5.0 | 12.0 | 2.5 |
| Hydrogenated coconut glycerides | 2.0 | 1.0 | 2.5 | 5.0 | 0.25 |
| Hydrogenated polyisobutene | 0.5 | 0.75 | 1.0 | 2.0 | 0.25 |
| Octyldodecanol | 0.5 | 1.0 | 0.75 | 3.0 | 0.25 |
| Aluminum stearate | 0.4 | 0.3 | 0.6 | 1.0 | 0.05 |
| Dicaprylyl carbonate | 0.1 | 0.05 | 0.15 | 0.5 | 1.0 |
| Hydrogenated castor oil | 0.5 | 0.75 | 1.0 | 2.5 | 5.0 |
| Sorbitol | 12.5 | 1.0 | 0.75 | 0.25 | 0.1 |
| Magnesium sulfate | 0.5 | 0.6 | 0.5 | 0.7 | 1.0 |
| Glycerol | — | 5.0 | — | 15.0 | 5.5 |
| Tetrasodium iminosuccinate | 1.5 | 0.6 | 3.0 | 0.4 | 1.0 |
| Perfume | q,s, | q,s, | q,s, | q,s, | q,s, |
| 1,3-Butylene glycol | — | — | 5.0 | — | 7.5 |
| Caprylic/capric acid triglyceride | 2.0 | 2.5 | 3.0 | 5.0 | 0.5 |
| Methyl paraben | 0.4 | 0.15 | 0.05 | 0.3 | 0.4 |
| Propyl paraben | 0.3 | 0.4 | 0.25 | 0.15 | — |
| Water | to 100 | to 100 | to 100 | to 100 | to 100 |

W/S emulsion

| | 11 | 12 | 13 | 14 | 15 |
|---|---|---|---|---|---|
| Cetyl dimethicone copolyol | 1.0 | — | — | 3.0 | 5.0 |
| Cyclomethicone + dimethicone copolyol | 10.0 | 12.5 | 25 | — | — |
| Cyclomethicone | 12.5 | 15 | 28.0 | 25.0 | 17.5 |
| Dimethicone | 5.0 | 13.0 | 5.0 | 12.0 | 15.0 |
| Hydrogenated polyisobutene | 0.5 | 0.75 | 1.0 | 2.0 | 0.25 |
| Octyldodecanol | 0.5 | 1.0 | 0.75 | 3.0 | 0.25 |
| Panthenol | 0.5 | 1.0 | 0.75 | 0.25 | 0.1 |
| Magnesium chloride | 2.0 | 0.6 | 2.5 | 0.7 | 1.0 |
| Glycerol | 25.0 | 5.0 | 10.0 | 15.0 | 57.5 |
| Tetrasodium iminosuccinate | 0.6 | 0.1 | 1.2 | 0.15 | 5.0 |
| Perfume | q,s, | q,s, | q,s, | q,s, | q,s, |
| Methyl paraben | 0.4 | 0.1 | 0.05 | 0.3 | 0.4 |
| Butylene glycol | — | 5.0 | — | — | 7.5 |
| Propyl paraben | 0.3 | 0.4 | 0.25 | 0.15 | — |
| Cetyldimethicone | 0.5 | — | 0.7 | — | — |
| Iodopropynyl butyl carbamate | — | — | 0.05 | — | 0.1 |
| Modified starch | — | 2.5 | — | 0.15 | — |
| Water | to 100 | to 100 | to 100 | to 100 | to 100 |

O/W emulsions

| | 16 | 17 | 18 | 19 | 20 |
|---|---|---|---|---|---|
| Glyceryl stearate citrate | 2 | — | — | — | — |
| Glyceryl stearate | — | 5 | 2 | 3 | — |
| PEG-40 stearate | — | — | 1 | — | — |
| Triglycerol methylglucose distearate | — | — | — | — | 3 |
| Sorbitan stearate | — | — | — | — | 1 |
| Cetearyl glucoside | — | — | — | 2 | — |
| Behenyl alcohol | — | — | — | — | 1 |
| Stearyl alcohol | 2 | 1 | — | — | — |
| Cetylstearyl alcohol | — | — | 2 | — | — |
| Hydrogenated coconut fatty glycerides | 2 | — | — | 1 | — |
| Shea butter | — | 2 | — | — | — |
| Butylene glycol dicaprylate/dicaprate | 1 | — | — | — | — |
| Caprylic/capric triglyceride | — | 4 | — | — | 1 |
| Ethylhexyl coconut fatty acid ester | 3 | — | — | — | — |
| Octyldodecanol | — | — | 5 | 8 | — |
| Mineral oil | 8 | 1 | — | — | 5 |
| Tetrasodium iminosuccinate | 1 | 0.5 | 2.5 | 0.3 | 0.75 |
| Vaseline | 4 | — | — | 2 | — |
| Octamethyltetrasiloxane | 5 | 1 | 3 | 1 | 3 |
| Dimethylpolysiloxane | — | 3 | 1 | 3 | 2 |
| Dicarprylyl carbonate | 10 | 1 | 8 | 5 | 2 |
| Glycerol | 3.0 | — | 25 | 12.5 | 30 |
| Butylene glycol | 18 | 15 | — | — | — |
| Methyl paraben | 0.3 | — | — | 0.2 | 0.4 |
| Iodopropynyl butyl carbamates | 0.1 | 0.2 | 0.2 | 0.05 | — |
| Perfume | q.s. | q.s. | q.s. | q.s. | q.s. |
| Water | to 100 | to 100 | to 100 | to 100 | to 100 |
| | 21 | 22 | 23 | 24 | 25 |
| Glyceryl stearate citrate | 5 | — | — | — | — |
| Glyceryl stearate | — | 5 | — | — | — |
| PEG-40 stearate | — | 2 | — | — | — |
| Polyethylene glycol (21) stearyl ether | — | — | 2 | — | — |
| Polyethylene glycol (2) stearyl ether | — | — | 1 | — | — |
| Cetearyl glucoside | — | — | — | 2 | — |
| Stearic acid | — | — | — | — | 2.5 |
| Behenyl alcohol | — | — | — | — | 2 |
| Stearyl alcohol | 2 | 1 | — | 5 | — |
| Cetylstearyl alcohol | — | — | 2 | — | 1 |
| Hydrogenated coconut fatty glycerides | 2 | — | — | 3 | 1 |
| Shea butter | — | 2 | — | 3 | — |
| Butylene glycol dicaprylate/dicaprate | 1 | — | 8 | — | 2 |
| Caprylic/capric triglyceride | — | 4 | 2 | — | — |
| Ethylhexyl coconut fatty acid ester | 3 | 6 | — | — | 2 |
| Octyldodecanol | — | — | 1 | 9 | — |
| Mineral oil | 9 | 1 | 1 | 1 | 3 |
| Vaseline | 4 | 2 | 5 | 0.5 | 0.75 |
| Glycerol | 7.5 | 15 | 65 | 25 | — |
| Sorbitol | 3.5 | — | — | — | 15 |
| Tetrasodium iminosuccinate | 1 | 0.5 | 2.5 | 1.25 | 0.75 |
| Octamethyltetrasiloxane | — | 1 | 2 | 5 | — |
| Dimethylpolysiloxane | 0.5 | 0.75 | 1.25 | — | 1 |
| Dicarprylyl carbonate | 6 | 2 | 10 | 0.5 | 4 |
| Methyl paraben | 0.3 | — | 0.1 | — | 0.05 |
| Iodopropynyl butyl carbamate | 0.1 | 0.2 | 0.1 | 0.2 | 0.15 |
| Perfume | q.s. | q.s. | q.s. | q.s. | q.s. |
| Water | to 100 | to 100 | to 100 | to 100 | to 100 |

That which is claimed:

1. A cosmetic or dermatological formulation, comprising
a) iminodisuccinic acid or a salt thereof; and
b) a polyol.

2. A cosmetic or dermatological formulation as claimed in claim 1, wherein the iminodisuccinic acid or a salt thereof is present in an amount of 0.001 to 15 wt. %, based on the total weight of the formulation.

3. A cosmetic or dermatological formulation as claimed in claim 1, wherein the iminodisuccinic acid or a salt thereof is present in an amount of 0.01 to 10 wt. %, based on the total weight of the formulation.

4. A cosmetic or dermatological formulation as claimed in claim 1, wherein the iminodisuccinic acid or a salt thereof is present in an amount of 0.05 to 5 wt. %, based on the total weight of the formulation.

5. A cosmetic or dermatological formulation as claimed in claim 1, wherein the polyol is present in a concentration of 3 to 65% by weight, based on the total weight of the formulation.

6. A cosmetic or dermatological formulation as claimed in claim 1, wherein the polyol is present in a concentration of 2 to 25% by weight, based on the total weight of the formulation.

7. A cosmetic or dermatological formulation as claimed in claim 1, wherein the iminodisuccinic acid or a salt thereof includes iminodisuccinic acid.

8. A cosmetic or dermatological formulation as claimed in claim 1, wherein the iminodisuccinic acid or a salt thereof includes the tetrasodium salt of iminodisuccinic acid.

9. A cosmetic or dermatological formulation as claimed in claim 1, wherein the polyol includes one or more polyols selected from the group consisting of glycerol, sorbitol and butylene glycol.

10. A cosmetic or dermatological formulation as claimed in claim 1, wherein the polyol includes glycerol, sorbitol and butylene glycol.

11. A skin care product including the cosmetic or dermatological formulation as claimed in claim 1.

12. A face care product including the cosmetic or dermatological formulation as claimed in claim 1.

13. A sunscreen product including the cosmetic or dermatological formulation as claimed in claim 1.

14. A method of moisturizing the skin, comprising applying to the skin a cosmetic or dermatological formulation comprising iminodisuccinic acid or a salt thereof; and a polyol.

15. A method as claimed in claim 14, wherein the iminodisuccinic acid or a salt thereof is present in the cosmetic or dermatological formulation in an amount of 0.001 to 15 wt. %, based on the total weight of the formulation.

16. A method as claimed in claim 14, wherein the iminodisuccinic acid or a salt thereof is present in the cosmetic or dermatological formulation in an amount of 0.01 to 10 wt. %, based on the total weight of the formulation.

17. A method as claimed in claim 14, wherein the iminodisuccinic acid or a salt thereof is present in the cosmetic or dermatological formulation in an amount of 0.05 to 5 wt. %, based on the total weight of the formulation.

18. A method as claimed in claim 14, wherein the polyol is present in the cosmetic or dermatological formulation in a concentration of 3 to 65% by weight, based on the total weight of the formulation.

19. A method as claimed in claim 14, wherein the polyol is present in the cosmetic or dermatological formulation in a concentration of 2 to 25% by weight, based on the total weight of the formulation.

20. A method as claimed in claim 14, wherein the iminodisuccinic acid or a salt thereof in the cosmetic or dermatological formulation includes iminodisuccinic acid.

21. A method as claimed in claim 14, wherein the iminodisuccinic acid or a salt thereof in the cosmetic or dermatological formulation includes the tetrasodium salt of iminodisuccinic acid.

22. A method as claimed in claim 14, wherein the polyol in the cosmetic or dermatological formulation includes one or more polyols selected from the group consisting of glycerol, sorbitol and butylene glycol.

23. A method as claimed in claim 14, wherein the polyol in the cosmetic or dermatological formulation includes glycerol, sorbitol and butylene glycol.

24. A method of increasing the skin-moisturizing action of a polyol in a cosmetic or dermatological formulation, comprising adding to the cosmetic or dermatological formulation iminodisuccinic acid or a salt thereof.
